(19) United States
(12) Patent Application Publication
(10) Pub. No.: US 2026/0191767 A1
(43) Pub. Date: Jul. 9, 2026

(54) ANTIMICROBIAL COMPOSITIONS

(71) Applicant: URGO US Inc., Fort Worth, TX (US)

(72) Inventors: Svetlana PANICHEVA, Downingtown, PA (US); Vadim PANICHEV, Downingtown, PA (US); Vincent PEYROU, Hauteville-lès-Dijon (FR); Jérémy CAPPE, Villers-Les-Pots (FR); Céline HENG-KISSENBERGER, NUITS-SAINT-GEORGES (FR)

(21) Appl. No.: 19/443,252

(22) Filed: Jan. 8, 2026

Related U.S. Application Data
(60) Provisional application No. 63/743,063, filed on Jan. 8, 2025.

(51) Int. Cl. A61K 9/00; A23B 2/729; A61K 31/19; A61K 33/20; A61K 47/02; A61P 31/04

(52) U.S. Cl. CPC A61K 9/0014; A23B 2/7295; A61K 31/19; A61K 33/20; A61K 47/02; A61P 31/04

(57) ABSTRACT

The disclosure provides an antimicrobial composition comprising hypohalous acid and a salt of an organic acid, as well as methods of use for disinfecting mammalian tissue, including wounds and burns, and other uses.

### PRIORITY

This application claims the benefit of, and priority to, U.S. Provisional Application No. 63/743,063 filed Jan. 8, 2025, which is hereby incorporated by reference in its entirety.

### BACKGROUND

Communities of microbes can adhere to a wound surface and form biofilm, a sticky protective layer which can significantly hinder wound healing by shielding microbial cells such as bacteria from immune response, promoting inflammation, and causing antibiotic resistance. Managing biofilm is crucial in treating chronic wounds, often requiring specific cleansing, debridement techniques, and antimicrobial dressings to disrupt and remove the biofilm effectively. Indeed, biofilm presence is strongly linked to delayed wound healing in chronic wounds, as it creates a barrier that prevents normal tissue regeneration and can lead to recurrent infections.

Antimicrobial compositions that are effective in the presence of biofilms and/or adherent microbial cells are needed. In the various aspects and embodiments, the present disclosure provides compositions for disinfection of various surfaces and materials, including but not limited to mammalian tissues, and which can be effective in the presence of biofilm and adherent microbial cells, and other environments that complicate disinfection.

### SUMMARY OF THE INVENTION

In aspects and embodiments, there is provided an antimicrobial composition for disinfection, including for surfaces and materials that may include biofilms and/or adherent microbial cells. In aspects and embodiments, the composition can be useful for disinfecting or treating mammalian or animal tissue, including but not limited to treating wounds (including chronic wounds) and burns. In aspects and embodiments, the compositions are useful for disinfecting or cleansing hard surfaces (including implantable or implanted medical devices), treating (e.g., preserving and/or disinfecting) food products, or controlling microbial contamination of water, among other uses.

In aspects and embodiments, there is provided an antimicrobial composition comprising hypohalous acid and a salt of an organic acid, such as an organic acid having from one to six carbon atoms, including but not limited to salts of short-chain fatty acids. In embodiments, the salt of a short-chain fatty acid is selected from a salt of one or more of acetate, propionate, butyrate, isobutyrate, valerate, isovalerate, and 2-methylbutanoate. In embodiments, the salt is a formate salt. In embodiments, the salt is an organic salt of active metals, such as one or more of alkaline, alkaline-earth, and aluminum metals. Exemplary metals include potassium, sodium, lithium, magnesium, calcium, and aluminum. In embodiments, the salt is an ammonium salt.

In embodiments, the salt is an acetate salt, such as one or more of potassium acetate, sodium acetate, lithium acetate, magnesium acetate, calcium acetate, aluminum acetate, and ammonium acetate. In embodiments, the acetate salt comprises potassium acetate. In embodiments, the acetate salt comprises sodium acetate. In embodiments, the acetate salt comprises aluminum acetate and/or lithium acetate.

In embodiments, the hypohalous acid is selected from hypochlorous acid and hypobromous acid. In embodiments, the hypohalous acid is hypochlorous acid.

In embodiments, the composition comprises available free chlorine (AFC) from about 50 to about 5,000 ppm. In embodiments, the AFC is from about 50 to about 1,000 ppm. In embodiments, the AFC is from about 50 to about 500 ppm, or from about 100 ppm to about 500 ppm. In embodiments, the AFC is from about 200 ppm to about 500 ppm, or from about 300 ppm to about 500 ppm, or from about 400 ppm to about 500 ppm.

In embodiments, the salt of an organic acid has a concentration from about 0.001 M to about 1.0 M. In embodiments, the salt of an organic acid has a concentration from about 0.005 M to about 0.1 M. In embodiments, the salt of an organic acid has a concentration from about 0.005 M to about 0.05 M, or from about 0.005 M to about 0.02 M. In embodiments, the salt of an organic acid has a concentration of about 0.005 M, about 0.01 M, or about 0.02 M, and wherein the salt optionally comprises an acetate salt such as potassium acetate and/or sodium acetate. In embodiments, the composition of the present disclosure comprises from about 0.005 M to about 0.02 M of the salt of an organic acid (e.g., comprising potassium acetate and/or sodium acetate) and from about 100 to about 500 ppm AFC (e.g., from about 400 to about 500 ppm) predominately in the form of hypochlorous acid.

In embodiments, the composition of the present disclosure comprises from about 0.005 M to about 0.02 M of the salt of an organic acid (e.g., potassium or sodium acetate), from about 100 to about 500 ppm hypochlorous acid (e.g., about 400 to about 500 ppm HOCl), and wherein the pH is about 3.5 to about 6.8. In embodiments, the pH is from about 3.8 to about 6.5. In embodiments, the pH is from about 4.0 to about 5.0. In embodiments, the pH is about 4.5.

In embodiments, the composition of the present disclosure further comprises a stabilizing amount of dissolved inorganic carbon (DIC). In embodiments, the DIC is incorporated in the form of a bicarbonate or carbonate of an alkali or alkaline earth metal. In embodiments, the composition of the present disclosure further comprises a stabilizing amount of sodium bicarbonate.

In embodiments, the composition is formulated as a solution, gel, cream, or foam. In embodiments, the composition comprises a gelling agent, such as lithium magnesium sodium fluorosilicate.

In aspects and embodiments, there is provided a method for preparing the composition of the present disclosure, comprising: incorporating the acetate salt into a hypohalous acid composition (e.g., HOCl solution), wherein the acetate salt is added to an electrolyte (e.g., comprising a halide salt) prior to electrolysis or added to a hypohalous acid solution after electrolysis.

In aspects and embodiments, there is provided a method for disinfecting or cleansing a mammalian or animal tissue, comprising applying the composition of the present disclosure to the mammalian or animal tissue, and where the tissue may comprise biofilms (e.g., bacterial and/or fungal biofilms). In embodiments, the mammalian or animal tissue is infected or colonized by bacterial or fungal species, and the tissue may comprise an injury such as a wound or a burn, or alternatively may comprise a dermatosis, such as atopic dermatitis. In embodiments, the composition is applied to an ocular surface or structure, including for the treatment or prevention of conjunctivitis or other ocular conditions characterized by infection and/or inflammation.

In aspects and embodiments, there is provided a method for disinfecting or cleansing a hard surface, comprising applying the composition of the present disclosure to the hard surface. In embodiments, the hard surface comprises porcelain, steel, titanium, alloys (e.g., titanium or chromium alloy, or Hastelloy), or ceramic material.

In aspects and embodiments, there is provided a method for preserving or decontaminating food products, comprising applying the composition of the present disclosure to a food product (e.g., vegetables, fruit, or meat).

In aspects and embodiments, there is provided a method for controlling microbial contamination of water, comprising applying the composition of the present disclosure to water in need of treatment or control for microbial contamination. In embodiments, the composition for controlling contamination of water comprises hypochlorous acid and/or hypobromous acid.

Other aspects and embodiments of the present disclosure will be apparent from the following disclosure and examples.

### DESCRIPTION OF THE FIGURES

FIGS. 1A-1B show graphs comparing concentration of S. aureus and P. aeruginosa (log₁₀ CFU/mL), respectively, after treatment with hypochlorous acid compositions after 1 minute of exposure.

FIG. 2A-2B show graphs comparing reduction of concentration of S. aureus and P. aeruginosa (log₁₀ CFU/mL), respectively, after treatment with hypochlorous acid compositions after 5 minutes of exposure.

FIG. 3 compares concentration of S. aureus (log₁₀ CFU/mL) after 1 and 5 minutes of exposure to hypochlorous acid compositions.

FIG. 4 compares concentration of P. aeruginosa (log₁₀ CFU/mL) after 1 and 5 minutes of exposure to hypochlorous acid compositions.

FIGS. 5A-5B show graphs comparing concentration of S. aureus and P. aeruginosa (log₁₀ CFU/mL), respectively, after treatment with saline and potassium acetate (KA).

FIG. 6 shows a graph measuring the concentration of S. aureus (log₁₀ CFU/cm²) after treatment with Formulation O, Comparative Formulation, KA and saline after 1 minute of exposure, followed by 24-hour incubation time.

FIG. 7 shows a graph measuring the concentration of S. aureus (log₁₀ CFU/cm²) after treatment with Formulation O, Comparative Formulation, KA and saline solutions after 5 minutes of exposure, followed by 24-hour incubation time.

FIG. 8 shows a graph measuring the concentration of P. aeruginosa (log₁₀ CFU/cm²) after treatment with Formulation O, Comparative Formulation, KA and saline after 1 minute of exposure, followed by 24-hour incubation time.

FIG. 9 shows a graph measuring the concentration of P. aeruginosa (log₁₀ CFU/cm²) after treatment with Formulation O, Comparative Formulation, KA and saline solutions after 5 minutes of exposure, followed by 24-hour incubation time.

FIG. 10 shows two graphs measuring the concentration of S. aureus (top) and P. aeruginosa (bottom) (log₁₀ CFU/mL) after treatment with KA after 1 minute, 5 minutes, and 15 minutes of exposure.

FIG. 11 shows a graph measuring the biofilm activity against MRSA biofilm (Log CFU/cm²) after single and repeated treatments of hypochlorous acid solutions, KA or saline solution.

FIG. 12A-12B shows two graphs measuring antibiofilm activity (log reduction CFU/mL) against S. aureus and P. aeruginosa (MRSA) after treatment with formulations containing 425-500 ppm and a buffer agent (potassium acetate KA or sodium acetate NaA) at concentrations of 0.005-0.01M, following a 15-minute exposure period.

FIG. 13 shows cytotoxicity potential of HOCl compositions comprising KA on mouse fibroblasts.

FIG. 14 shows cytotoxicity potential of HOCl compositions comprising KA or NaA on mouse fibroblasts.

### DETAILED DESCRIPTION

In aspects and embodiments, there is provided an antimicrobial composition comprising hypohalous acid and a salt of an organic acid. In various embodiments, the pH of the composition is from about 3.5 to about 6.8. The antimicrobial composition is useful for disinfecting surfaces and materials (including organic and inorganic materials), including surfaces and materials that may include biofilms and/or adherent bacterial and/or fungal cells. For example, the composition can be useful for disinfecting or treating mammalian or animal tissues, including but not limited to treating wounds (including chronic wounds) and burns. The compositions are further useful for disinfecting or cleansing hard surfaces, preserving and/or disinfecting food products, or controlling microbial contamination of water, among other uses. In various embodiments, the antimicrobial compositions show good stability, low cytotoxicity, and high biocidal properties.

In aspects and embodiments, there is provided an antimicrobial composition comprising hypohalous acid and a salt of an organic acid, such as a salt of an organic acid having from one to six carbon atoms, including but not limited to salts of short-chain fatty acids. In embodiments, the salt of a short-chain fatty acid is selected from a salt of one or more of acetate, propionate, butyrate, isobutyrate, valerate, isovalerate, and 2-methylbutanoate. Alternatively, the salt is a formate salt. In embodiments, the salt is an organic salt of active metals, such as one or more of alkaline, alkaline-earth, and aluminum metals. Exemplary metals include potassium, sodium, lithium, magnesium, calcium, and aluminum. In embodiments, the salt is an ammonium salt.

In embodiments, the salt is an acetate salt, such as an acetate salt comprising one or more of potassium acetate, sodium acetate, lithium acetate, aluminum acetate, ammonium acetate, magnesium acetate, and calcium acetate. In embodiments, the acetate salt comprises potassium acetate. In embodiments, the acetate salt comprises sodium acetate. In embodiments, the acetate salt comprises aluminum acetate and/or lithium acetate.

In embodiments, the hypohalous acid is selected from hypochlorous acid (HOCl) and hypobromous acid (HOBr). In embodiments, the hypohalous acid is hypochlorous acid. In embodiments, the hypohalous acid solution is generated by electrolysis of a halide salt, such as sodium chloride. In embodiments, the hypohalous acid solution comprises a mixture of oxidizing species (e.g., Available Free Chlorine, or AFC) such as predominantly hypochlorous acid, sodium hypochlorite, and molecular chlorine (Cl₂), the relative amounts of which will depend on the pH. In embodiments, the AFC comprises or consists essentially of hypochlorous acid. In embodiments, the antimicrobial composition comprises other hypohalous acids such as hypobromous acid, or mixture thereof. In some embodiments, the solution contains or forms other oxidizing or radical producing species such as a hypohalite (e.g., hypochlorite), hydroxide, H₂O₂ and O₃, among others.

The biocidal activity of the solution can be expressed in terms of available free chlorine (AFC). As used herein, the AFC of a composition is the initial AFC of the composition when it is made (e.g., by electrolysis of a halide salt). In other aspects referring to methods of use, the AFC refers to the AFC of the composition when it is applied to a substrate, e.g., for purposes of disinfection. In embodiments, the composition comprises an AFC from about 50 to about 5,000 ppm. In embodiments, the AFC is from about 50 to about 1,000 ppm. In embodiments, the AFC is from about 50 to about 500 ppm, or from about 100 ppm to about 500 ppm, or from about 300 ppm to about 500 ppm, or from about 400 ppm to about 500 ppm. In embodiments, the AFC is about 100 ppm, or about 150 ppm, or about 200 ppm, or about 250 ppm, or about 300 ppm, or about 400 ppm, or about 425 ppm, or about 450 ppm, or about 500 ppm. In embodiments, AFC levels can be selected based on intended application.

While the hypochlorous acid may be produced chemically in accordance with some embodiments (e.g., by acidification of hypochlorite or injection of molecular chlorine into alkaline solutions, including hydroxides, carbonates or bicarbonates of alkaline or alkaline-earth metals), the hypohalous acid may also be produced electrochemically. The electrochemical production of hypohalous acid is by treatment of halide-based electrolytes in a diaphragm-type electrolytic cell. Electrochemical treatment of saline is described, for example, in U.S. Pat. Nos. 5,427,667, 5,635,040, 5,783,052, 7,303,660, 7,828,942, 7,897,023, and 8,961,750, which are hereby incorporated by reference in their entireties.

In embodiments, the salt of an organic acid has a concentration from about 0.001 M to about 1.0 M, or from about 0.002 M to about 0.5 M. In embodiments, the salt of an organic acid has a concentration from about 0.005 M to about 0.1 M. In embodiments, the salt of an organic acid has a concentration from about 0.005 M to about 0.05 M. In embodiments, the salt of an organic acid has a concentration of from about 0.005 M to about 0.02 M, such as about 0.005 M, about 0.01 M, or about 0.02 M. In embodiments, the salt of an organic acid has a concentration of from about 0.005 M to about 0.02 M, or a concentration of from about 0.005 M to about 0.01 M, and optionally the salt is an acetate salt which optionally comprises potassium acetate and/or sodium acetate. In embodiments, the composition of the present disclosure comprises from about 0.005 M to about 0.02 M (or about 0.005 M to about 0.01M) of the salt of an organic acid (e.g., comprising potassium acetate and/or sodium acetate) and from about 100 to about 500 ppm hypochlorous acid (e.g., about 400 to about 500 ppm HOCl). In embodiments, the composition of the present disclosure comprises from about 0.005 M to about 0.02 M of the salt of an organic acid (e.g., comprising potassium acetate and/or sodium acetate) and from about 100 ppm to about 500 ppm hypochlorous acid, such as about 150 ppm, or about 200 ppm, or about 250 ppm, or about 300 ppm, or about 350 ppm, or about 400 ppm, or about 425 ppm, or about 450 ppm, or about 500 ppm hypochlorous acid. In embodiments, the combination of hypochlorous acid and the salt of an organic acid increases the biocidal activity (including in the presence of biofilms or adherent bacterial or fungal cells, or combination of both) at relatively low AFC.

In embodiments, the pH of the composition is from about 3.8 to about 6.5. In embodiments, the pH of the composition is from about 4.0 to about 6.0, or from about 4.0 to about 5.0. In embodiments, the pH of the composition is about 4.5. As used herein, the pH of a composition is the initial pH of the composition when it is made (e.g., by electrolysis of a halide salt and addition of acetate salt). In other aspects referring to methods of use, the pH refers to the pH of the composition when it is applied for purposes of disinfection.

In embodiments, the composition of the present disclosure comprises from about 0.005 M to about 0.05 M potassium acetate, from about 100 to about 500 ppm hypochlorous acid (as already described), and a pH as described in this paragraph. In embodiments, the composition of the present disclosure comprises from about 0.005 M to about 0.02 M potassium acetate (e.g., 0.005 M to about 0.01 M potassium acetate), about 100 to 500 ppm of hypochlorous acid (e.g., from about 400 to 500 ppm HOCl), and wherein the pH is about 4.5. In embodiments, the composition of the present disclosure comprises from about 0.005 M to about 0.02 M potassium acetate, about 100 ppm, about 150 ppm, about 200 ppm, about 250 ppm, or about 300 ppm, or about 350 ppm, or about 400 ppm, or about 450 ppm, or about 500 ppm of hypochlorous acid, and wherein the pH is about 4.5. In the context of pH, the term “about” means±0.2.

In embodiments, the composition of the present disclosure comprises from about 0.005 M to about 0.05 M sodium acetate, from about 100 to about 500 ppm hypochlorous acid (as already described), and a pH as described in this paragraph. In embodiments, the composition of the present disclosure comprises from about 0.005 M to about 0.02 M sodium acetate (e.g., 0.005 M to about 0.01 M sodium acetate), about 100 to 500 ppm of hypochlorous acid (e.g., from about 400 to 500 ppm HOCl), and wherein the pH is about 4.5. In embodiments, the composition of the present disclosure comprises from about 0.005 M to about 0.02 M sodium acetate, about 100 ppm, about 150 ppm, about 200 ppm, about 250 ppm, or about 300 ppm, or about 350 ppm, or about 400 ppm, or about 450 ppm, or about 500 ppm of hypochlorous acid, and wherein the pH is about 4.5. In the context of pH, the term “about” means±0.2.

In embodiments, the composition of the present disclosure further comprises a stabilizing amount of dissolved inorganic carbon (DIC), as described in U.S. Pat. No. 10,576,152, which is hereby incorporated by reference in its entirety. In embodiments, the DIC is incorporated in the form of a bicarbonate or carbonate of an alkali or alkaline earth metal. In embodiments, the composition of the present disclosure comprises a stabilizing amount of sodium bicarbonate.

In embodiments, the amount of sodium bicarbonate to AFC ratio of the composition is at least about 1:2. In embodiments, the amount of sodium bicarbonate to AFC ratio is from about 5:1 to about 1:5. In embodiments, the amount of sodium bicarbonate is about equimolar with the AFC content. In embodiments, the carbonate or bicarbonate is contained within an electrolyte or added to an electrolyzed solution. In embodiments, the AFC and the pH of the composition are stable for at least 6 months, or stable for at least 9 months, or stable for at least one year. For example, in embodiments, the composition retains at least 50% of the initial AFC for at least 6 months, or at least 9 months, or at least one year. In embodiments, the pH of the composition is stable (e.g., does not vary by more than 0.5 from an initial pH) for at least 6 months, or at least 9 months, or at least one year.

In embodiments, the composition is formulated as a solution, gel, cream, or foam. In embodiments, the composition comprises a gelling agent, which in embodiments comprises lithium magnesium sodium fluorosilicate. In embodiments, the composition may take the form of a cream, gel (e.g. silicon-based gel), and/or foam by the addition of conventional ingredients known in the art. For example, topical formulations of electrochemical solutions are disclosed in US 2005/0196462 and U.S. Pat. No. 9,925,217, which are hereby incorporated by reference in its entirety. In embodiments, the antimicrobial composition is better contained around the application site by limiting solution run-off. In embodiments, convenient applicators for creams, foams, and the like are known, and may be used in accordance with the present invention.

The composition may comprise pharmaceutically acceptable carriers known in the art. Non-limiting examples of suitable carriers include hectorite, bentonite, laponite, oil emulsions, polyvinyl alcohol, povidone, hydroxypropyl methyl cellulose, poloxamers, carboxymethyl cellulose, hydroxyethyl cellulose, and purified water. The composition may also include various other ingredients, such as tonicity agents, buffers, surfactants, co-solvents, viscosity building agents, and preservatives.

Regarding tonicity agents, such agents may be employed to adjust the tonicity of a composition, for example, in the case of an ophthalmic composition, to the tonicity of natural tears. For example, sodium chloride, potassium chloride, magnesium chloride, calcium chloride, dextrose and/or mannitol may be added to the composition to approximate physiological tonicity. Such an amount of tonicity agent will vary, depending on the particular agent to be added and the type of composition. In general, however, the compositions will have a tonicity agent in an amount sufficient to cause the final composition to have an acceptable osmolality. For example, the composition may have an osmolality in the range of about 150 to 450 mOsm, such as 250 to 350 mOsm.

Regarding buffers, an appropriate buffer system (such as, for example, sodium phosphates, potassium phosphates, potassium carbonate, sodium bicarbonate, sodium borate or boric acid) may be added to the compositions, including for gel or foam formulation, to prevent pH drift under storage conditions. The particular concentration will vary, depending on the agent employed. The buffer will generally be chosen to maintain a target pH within a range as described herein. In embodiments, the composition does not contain an additional buffer system, beyond the salt of organic acid (e.g., acetate salt as described).

Regarding a surfactant, various surfactants useful in conventional topical formulations may be employed. Exemplary surfactants include CREMOPHOR EL, lauramine oxide, myristyl dimethylamine oxide, polyoxyl 20 ceto stearyl ether, polyoxyl 40 hydrogenated castor oil, polyoxyl 23 lauryl ether and poloxamer 407.

Regarding viscosity building agents, such agents may be added to compositions to increase the viscosity of the carrier. Examples of viscosity enhancing agents include, but are not limited to: synthetic silicates, polysaccharides (e.g., hyaluronic acid, cellulose, and chitosan, and their salts), chondroitin sulfate and its salts, dextrans, various derivatives of the cellulose family (e.g., HPMC); vinyl polymers; and acrylic acid polymers. For example, the composition may exhibit a viscosity of 1 to 400,000 centipoises (“cps”).

Regarding preservatives, no additional antimicrobial agent is required, since the hypohalous acid will function as a preservative; however, in some embodiments a second preservative or antimicrobial agent is employed such as silver.

In embodiments, the composition consists essentially of or consists of the hypohalous acid and acetate salt, and optionally sodium bicarbonate (each as already described). In this context, the term consists essentially of means that other components can be included, as long as they are stable in the presence of the hypohalous acid (e.g., HOCl), and the stability of the hypohalous acid and stability of the pH of the composition are not significantly impacted.

In embodiments, the composition is shelf-stable and has the desired physical characteristics for topical treatment of skin (e.g., for topical treatment of a wound or burn). For example, hypochlorous acid solution can be used as a dispersing media with a silicate carrier to prepare a hydrogel. For example, the composition may be a hydrogel having a conductivity of from about 0.5 mS/cm to about 12 mS/cm, such as from about 1 mS/cm to about 10 mS/cm in some embodiments. The hydrogel may be prepared from silicate-based carriers, such as about 0.5% to about 5% of a fluorosilicate-based carrier, and may employ additional agents for targeting and maintaining the pH, such as phosphoric acid and sodium bicarbonate.

In some embodiments, the composition is a hydrogel employing a fluorosilicate-based carrier. The formulation may have a viscosity of from about 500 to about 1,500,000 cP, such as from about 1000 to about 150,000 cP, or from about 2,000 to about 40,000 cP. The formulation in some embodiments has a conductivity of less than 10 mS/cm, such as from about 0.5 to about 5 mS/cm, or from about 0.5 to about 3 mS/cm, or about 1 or about 2 mS/cm in some embodiments.

In aspects and embodiments, there is provided a method for preparing the composition of the present disclosure, comprising: incorporating the salt of an organic acid into a hypohalous acid composition. In embodiments, the acetate salt is added to an electrolyte prior to electrolysis, or alternatively, the salt of an organic acid is added to the composition after electrolysis. In embodiments, the antimicrobial composition of the present disclosure is hypertonic, hypotonic, or isotonic with respect to physiological fluids (blood, plasma, tears, etc.). In embodiments, the antimicrobial composition of the present disclosure contains varying levels of salinity, such as from 0.01 to about 2.0%. In embodiments, the electrolyzed solution is generated using a mixture of physiologically balanced salts, as disclosed in U.S. Pat. No. 6,426,066, which is hereby incorporated by reference in its entirety. Such salts may include potassium halides (e.g., KCl) and magnesium halides (e.g., MgCl₂), in addition to sodium halides (e.g., NaCl). Devices for electrolysis are known and described, for example, in U.S. Pat. Nos. 5,427,667, 5,635,040, 5,783,052, 7,303,660, 7,828,942, 7,897,023, and 8,961,750, which are hereby incorporated by reference in their entireties.

In aspects and embodiments, there is provided a method for disinfecting or cleansing a mammalian or animal tissue, comprising applying the composition of the present disclosure to the mammalian or animal tissue. In some embodiments, the tissue is a human tissue. In embodiments, the AFC of the applied composition is from about 50 to about 500 ppm, or from about 50 to about 400 ppm, or from about 50 ppm to about 300 ppm, or from about 50 ppm to about 200 ppm, or from about 200 to 500 ppm, or from about 300 to about 500 ppm, or from about 400 to 500 ppm. In embodiments, the AFC of the applied composition is about 50 ppm, about 100 ppm, about 150 ppm, about 200 ppm, about 250 ppm, or about 300 ppm, or about 350 ppm, or about 400 ppm, or about 425 ppm, or about 450 ppm, or about 500 ppm. In embodiments, the pH of the applied composition ranges from about 4.0 to about 6.5, such as from about 4.0 to about 6.0, or from about 4.0 to about 5.5, or from about 4.0 to about 5.0 (e.g., about 4.5).

In embodiments, the tissue is infected or colonized by bacterial or fungal pathogens, or in embodiments comprises a wound or a burn, a dermatosis (e.g., atopic dermatitis). In some embodiments, the wound is an acute or chronic wound. In embodiments, the antimicrobial composition of the present disclosure is used as a sterilizing, disinfecting, and/or debriding solution for human and animal wound care. For example, the method of the present disclosure provides for moistening, lubricating, irrigating, cleaning, deodorizing, disinfecting, or debriding a wound by rinsing, washing or immersing the wound, with or in, the composition (e.g., solution), or by applying the composition (e.g., as a solution) to the wound and/or wound dressing. In embodiments, the method of the present disclosure provides for rinsing a wound (e.g., pouring the composition over the wound). In embodiments, the method of the present disclosure provides for soaking a wound (e.g., soaked compress). In embodiments, the method of the present disclosure provides for instillation (e.g., instillation cycles).

In embodiments, the antimicrobial composition of the present disclosure provides a convenient means for wound care and management and is used in combination with the apparatus and methods described in U.S. 2010/030132, which is hereby incorporated by reference in its entirety. For example, the method comprises supplying the composition to a wound site by one or more of soak, scrub, pulsed lavage, hydrosurgery, and ultrasound to effectively debride and disinfect a wound or tissue. In embodiments, the antimicrobial composition of the present disclosure is delivered before, during and/or after negative pressure wound therapy to promote proper wound healing physiology. In embodiments, the present disclosure provides a method for treating an infected or colonized wound, tissue, surgical cavity, or bone, and a method for reducing wound bioburden. In embodiments, the antimicrobial composition of the present disclosure is generally effective for killing or inactivating a broad spectrum of bacterial, fungal, and viral pathogens, including S. aureus. In embodiments, the microbe is an antibiotic resistant bacterial or fungal pathogen, including but not limited to MRSA (Methicillin-Resistant S. aureus), ORSA (Oxacillin-resistant S. aureus), VRSA (Vancomycin-Resistant S. aureus), P. aeruginosa, E. coli (including CRE, or Carbapenem-resistant E. coli), Enterococcus spp. (including VRE, or Vancomycin-Resistant Enterococci), C. difficile, and Candida Spp. In embodiments, the antimicrobial composition of the present disclosure is generally effective for killing Gram positive and Gram negative bacterial and fungal pathogens. In embodiments, the antimicrobial composition of the present disclosure is generally effective for treating ulcers. In embodiments, the antimicrobial composition of the present disclosure is generally effective for treating chronic leg wounds. In embodiments, the antimicrobial composition of the present disclosure is generally effective for treating venous leg ulcers.

In embodiments, the antimicrobial composition of the present disclosure is useful for the control of inflammation, including an inflammatory reaction or hyper inflammation of the skin, mucosal membrane, or ocular surface. For example, the composition may be applied for use in a method as described in U.S. Pat. No. 10,632,147 or U.S. Pat. No. 10,987,379, which are hereby incorporated by reference. In certain embodiments, the composition of the invention is applied (e.g., to an effected area) for treatment of a patient having a dermatoses, atopic dermatitis, skin allergy, rosacea, psoriasis, or acne, among others. In such embodiments, the composition may be formulated as a hydrogel, for example, as described elsewhere herein.

In embodiments, the composition can be applied to affected areas of the skin, which may be characterized by an alkaline pH as compared to normal healthy skin. In such embodiments, the acidic pH of the composition helps bring the skin to a pH that is more conducive to healing and healthy regeneration. In some embodiments, a more alkaline skin is associated with over-colonization of certain microbes (e.g., Staphylococcus spp.), whereas a slightly acidic pH is more conducive to a healthy skin microbiome. Further, in some embodiments, application to intact but inflamed skin promotes healthy skin regeneration and barrier integrity, by inhibiting or reducing the tissue-damaging inflammatory response, thereby allowing the cells (e.g., dermal fibroblasts and/or keratinocytes) to proliferate in a manner consistent with the healing process. In embodiments, the composition is not cytotoxic to these cells at the levels applied. The healing environment is further aided by reducing the microbial burden of the inflamed tissue, where otherwise infection might spawn due to loss of barrier integrity. Thus, in various embodiments, the composition results in one or more of a reduction of microbial burden, a reduction of inflammation, altered or balanced skin and/or systemic immunity, reduced pruritis, enhanced skin cell regeneration, and normalized skin pH.

In various embodiments, the invention provides for administering the compositions to one or more tissues affected by infection and/or inflammation, including by topical application or as an irrigant to one or more tissues or organs (e.g., during surgery or after trauma). For example, in various embodiments, the affected tissue is one or more of the skin, eyes, ears, nose, sinus, throat, or mouth (e.g., gingiva). In other embodiments, the affected tissue is one or more of the intestinal tract and/or colon, lungs, or urogenital system (urinary tract or vagina).

In certain embodiments, the tissue comprises one or more structure of the eye, such as those affecting the conjunctiva, uvea, eye lids, oil glands, and lacrimal ducts. In embodiments, the composition is applied to treat bacterial, viral, or allergic conjunctivitis, uveitis, blepharitis, external and internal hordeolum, canaliculitis, dacrocystitis, and chalazions. In embodiments, the composition may be applied to treat various conditions affecting the ear (including the inner ear, middle ear, ear canal, and ear drum), nose, mouth and throat, including rhinitis, sinusitis, rhinorrhea, otitis media, external otitis, myringitis, pharyngitis, and stomatitis. In embodiments, the composition is useful as an alternative or adjunct therapy to conventional antibiotics, antivirals, decongestants, antihistamines, immunosuppressants/immunomodulators, analgesics/anesthetics and steroid treatments, or as an alternative to therapy using a combination of conventional medicaments. In embodiments, the compositions are particularly suitable for prolonged and/or prophylactic use, because of the associated age restrictions, side effects, and/or drug resistance issues with alternative therapies. The methods are further suitable for individuals prone to such infections and/or inflammatory conditions, or individuals that typically experience hypersensitivity or severe side effects with other treatments.

In certain embodiments, the composition can be employed to treat a condition involving a bacterial infection that produces a discharge, such as but not limited to bacterial conjunctivitis. In embodiments, the condition may result from or comprise a blocked gland or chronic inflammatory condition, including styes and chalazions, which may also develop an acute bacterial infection. Such conditions may be recurring or may be difficult to completely clear. In embodiments, cleaning the eye and the surrounding areas with the composition provides the benefit of effectively cleaning debris, biofilm, and/or discharge from the eye in a manner that reduces the risk of spreading the infection to an unaffected eye or another individual.

In certain other embodiments, the composition is administered prophylactically, especially where eye infections are likely to occur or be transmitted among persons. Such prophylactic care might include routine cleaning of the eyes and surrounding areas, such as the eyelids, with the composition, or routine rinsing of contact lenses by applying the composition to the contact lenses to decontaminate and clear debris and biofilm. Such prophylactic care might also include routine cleaning of the ear, mouth, throat, nose, or sinuses, with the composition. Such embodiments can result in the prevention of an infection, can prevent the worsening of an existing infection, or can prevent irritation caused by bacterial biofilm.

In other embodiments, the composition is applied to the environment via fogging, misting, or humidifying, to prevent the transfer of pathogens in air droplets.

In aspects and embodiments, there is provided a method for disinfecting or cleansing a hard surface, comprising applying the composition of the present disclosure to the hard surface. In embodiments, the hard surface comprises porcelain, steel, titanium, alloys (e.g., titanium alloy, chromium alloy, nickel alloy, or Hastelloy), or ceramic material. In embodiments, the surface is of an implantable or implanted medical device, including but not limited to artificial joints (e.g., hips and knees).

In aspects and embodiments, there is provided a method for preserving or decontaminating food products or plant products, comprising applying the composition of the present disclosure to a food product or plant product. Exemplary food products include vegetables, fruit, and meats, where decontamination may be necessary. In embodiments, the composition is applied to a plant product, including crops (pre- or post-harvest) or ornamental flowers (e.g., cut flowers).

In aspects and embodiments, there is provided a method for controlling microbial contamination of water, comprising applying the composition of the present disclosure to water in need of treatment or control for microbial contamination. In embodiments, compositions for controlling contamination of water comprise hypochlorous acid or hypobromous acid.

As used herein, the term “about” means±10% of a reference value, unless the context clearly requires otherwise.

### EXAMPLES

### Example 1: Evaluation of the Antibacterial Effect of Potassium Acetate (KA) and Hypochlorous Acid (HOCl) on Planktonic Cultures of Methicillin-Resistant Staphylococcus aureus (MRSA) and P. aeruginosa

The effect of KA and HOCl on bacterial strains (MRSA and Pseudomonas aeruginosa) was investigated, as well as a potential synergistic effect. The test solutions included: a solution of 0.02 M KA, a solution of 0.01 M KA, five test solutions containing HOCl at either 150, 300 or 425 ppm of available free chlorine (AFC) and KA at either 0.01 or 0.02 M (Formulation O), and three comparator solutions of HOCl containing either 150, 300 or 425 ppm of AFC (Comparative Formulation). The Solutions are summarized in Table 1 below.

| Solutions | AFC (ppm) | KA (M) | pH |
|---|---|---|---|
| KA | 0 | 0.01 | 4.5 |
| | 0 | 0.02 | 4.5 |
| Formulation O | 150 | 0.01 | 4.5 |
| | 150 | 0.02 | 4.5 |
| | 300 | 0.01 | 4.5 |
| | 300 | 0.02 | 4.5 |
| | 425 | 0.01 | 4.5 |
| Comparative Formulation | 150 | 0 | 4.5 |
| | 300 | 0 | 4.5 |
| | 425 | 0 | 4.5 |

Antibacterial effect on planktonic cultures of two reference strains, S. aureus (ATCC 43300 Methicillin-resistant strain) and P. aeruginosa (ATCC 9027) was evaluated. Staphylococcus is Gram positive while Pseudomonas is Gram negative.

### First Experimental Protocol

The test mix comprised 0.8 mL tested solution or saline solution, 0.1 mL bacterial suspension (10⁸ CFU/mL), 0.1 mL interfering substance (Simulated Wound Fluid, or SWF, comprising 50% v/v fetal calf serum and 50% v/v/maximum recovery diluent). The contact times were 15 seconds, 5 minutes, or 15 minutes. The neutralization step involved mixing 0.1 mL test mix and 0.9 mL neutralizer, for 5 minutes. The impact of the different tested solutions on cell viability in the bacterial suspension was assessed in comparison with a treatment with saline solution. Agar plates were incubated at 37° C. for 24 to 48 hours for the recovery of stressed cells. The neutralizer composition comprises a solution of sodium thiosulfate, with phosphatidylcholine, L-Histidine, and Polysorbate 80.

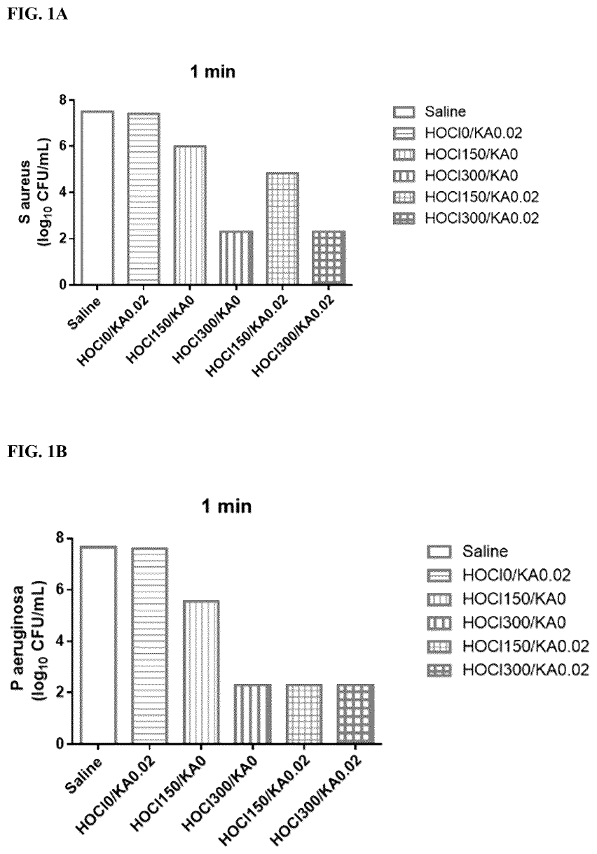

Results showed that there was no effect with KA alone, there was full anti-bacterial activities after exposure to Formulation O and Comparative Formulations at 300 ppm AFC with and without KA, and partial effect of anti-bacterial activities after exposure to Formulation O and Comparative Formulations with 150 ppm AFC, and 1.17 extra log reduction in the presence of KA (FIG. 1A-1B). The results suggest some synergy of HOCl and acetate salt at 150 ppm AFC.

A test was performed on S. aureus (MRSA) and P aeruginosa with formulation O and a comparative formulation at 425 ppm AFC with a contact time of 15 seconds; this test led to a full anti-bacterial activity for both formulations.

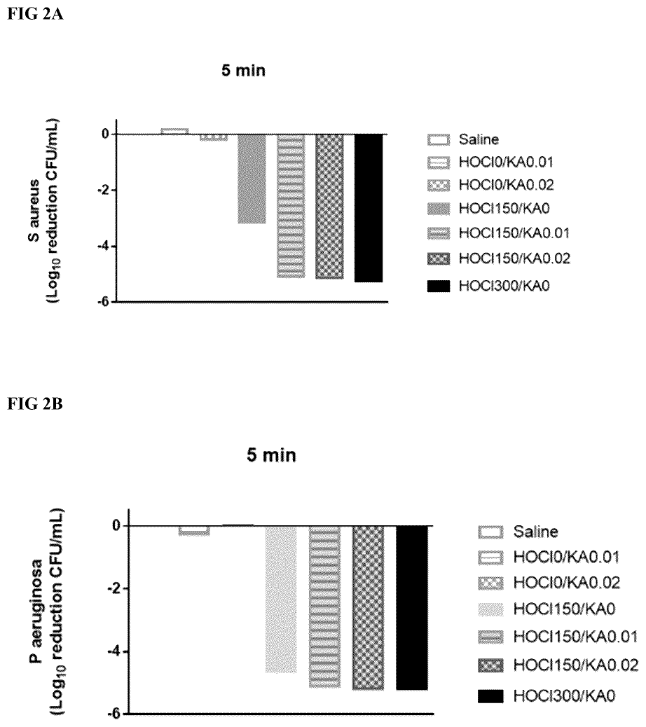

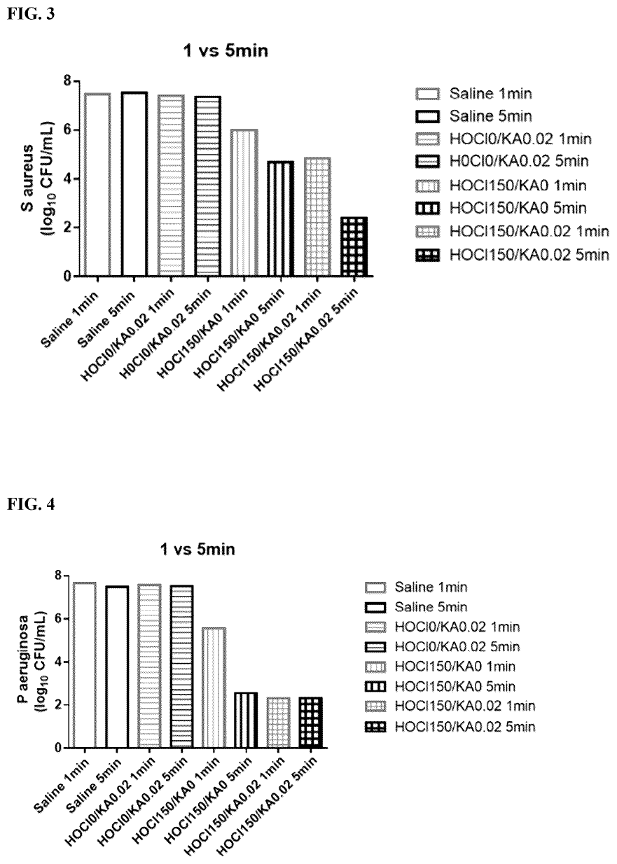

FIGS. 2A and 2B show the activity of tested solutions against S. aureus (MRSA) and P. aeruginosa after 5 min of contact. Results showed that, similarly to saline, there was no significant bactericidal effect of KA at 0.01 or 0.02M against MRSA and P. aeruginosa planktonic cells. Results showed that KA at either 0.01 or 0.02M strongly improved the antibacterial effect of HOCl. The formulations O at 150 ppm AFC provides the same antibacterial activity as the comparative formulation at 300 ppm with a total bacterial eradication.

FIG. 3 shows the activity of tested solutions against S. aureus (MRSA). Results showed that there was no significant bactericidal effect of KA against MRSA planktonic cells. The comparison of the effects after 1 and 5 minutes of exposure showed that KA strongly improved the antibacterial effect of HOCl and the kinetic of the antibacterial effect. A similar bactericidal effect was observed after 1 minute of contact with the formulation O at 150 ppm AFC than after 5 minutes of contact with the comparative formulation at 150 ppm AFC. After 5 min of contact, a full anti-bacterial activity was observed with the formulation O at 150 ppm AFC compared to a partial anti-bacterial activity with the comparative formulation at 150 ppm AFC.

FIG. 4 shows the activity of tested solutions against P. aeruginosa. Results showed that there was no bactericidal effect of KA against P. aeruginosa planktonic cells. The comparison of the effects after 1 and 5 minutes of exposure showed that KA strongly improved the antibacterial effect of HOCl and the kinetic of antibacterial effect. A similar bactericidal effect was observed after 1 minute of contact with the formulation O at 150 ppm AFC than after 5 minutes of contact with the comparative formulation at 150 ppm AFC. After 1 min of contact, a full anti-bacterial activity was observed with the formulation O at 150 ppm AFC compared to a partial anti-bacterial activity with the comparative formulation at 150 ppm AFC.

### Second Experimental Protocol

9.0 mL KA solution or saline solution were exposed to 1.0 mL bacterial suspension (10⁸ CFU/mL) for 15 min. The mixture was centrifuged for 5 minutes at 7,000 g and the supernatant was removed. The bacterial pellet was inoculated with 10 mL tryptic soy broth (TSB)/simulated wound fluid (SWF) and vortexed for 15 seconds and incubated at 37° C. Bacterial cells were counted immediately after exposure and after 1, 2, 3, 4, 5 and/or 6 hours of incubation. Agar plates were incubated at 37° C. for 24 to 48 hours for the recovery of stressed cells.

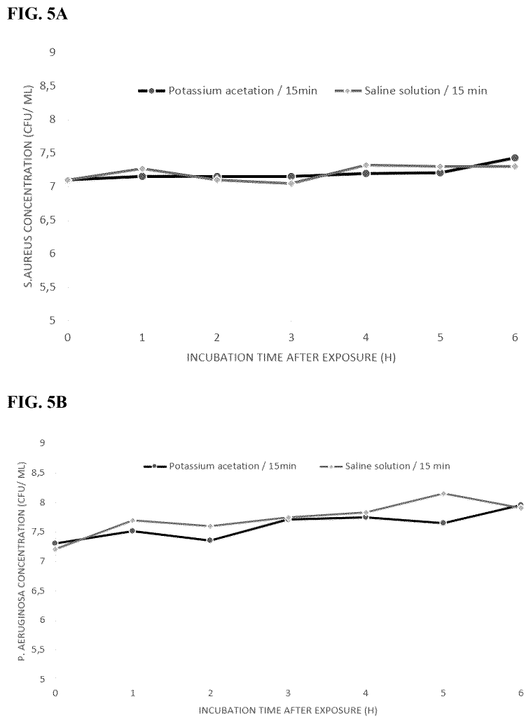

FIGS. 5A-5B show no significant effect of KA on the regrowth of P. aeruginosa and S. aureus MRSA planktonic cells after 15 minutes of exposure.

### Example 2: Evaluation of the Effect of Potassium Acetate on Adherent Cells Formation

### Adherent Cells Prevention Model Protocol

Bacterial suspension was prepared in SWF (10⁵ CFU/mL) and 0.5 mL of the preparation was inoculated on Collagen I-coated microplates and incubated at 37° C. for 2 hours. The medium was removed from the microplate and adherent cells were washed with saline solution. 0.5 mL of tested solutions or saline solution (control) was added. The microplate was incubated at room temperature for 1 or 5 minutes. The solutions were removed and the adherent cells were washed twice with saline solution. 0.5 mL of sterile SWF was added and the microplate was incubated at 37° C. The progress of adherent cells formation was monitored. Adherent bacterial cells or sessile cells were counted before and immediately after exposure, and after 2, 4, 6 and 24 hours of incubation. Agar plates were incubated at 37° C. for 24 to 48 hours for the recovery of stressed cells.

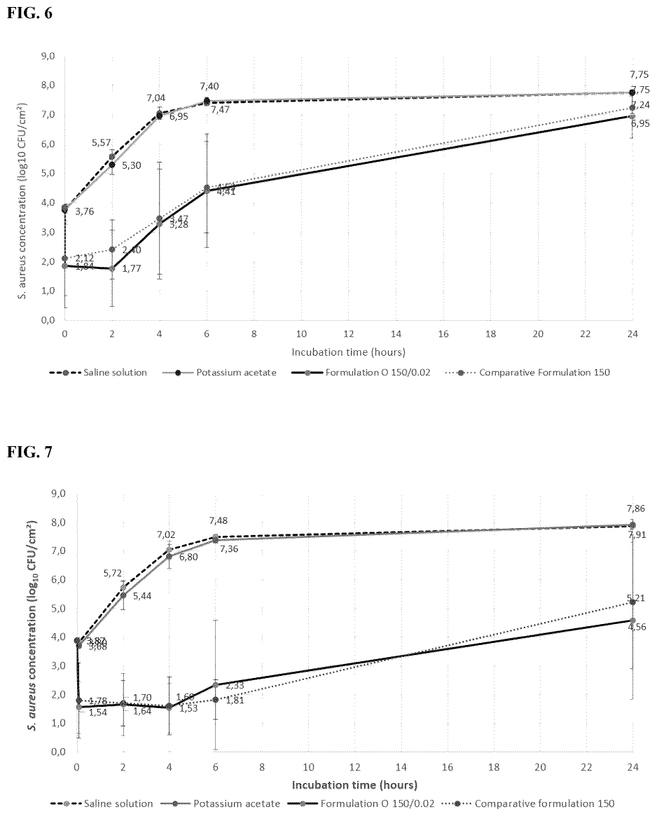

FIG. 6 shows the activity against S. aureus MRSA after 1 minute of exposure. Data showed no significant effect of KA against S. aureus (MRSA) adherent cells. There was strong effect of Formulation O and Comparative Formulations containing 150 ppm AFC on adherent cells after 1 minute of exposure. Regrowth was observed two to four hours after treatment with Formulation O and Comparative Formulations. After 1 minute of exposure to Formulation O and Comparative Formulations, the concentration of adherent bacterial cells (sessile cells) measured after 24 h were similar, but lower than those obtained after the treatment with saline solution.

FIG. 7 shows the activity against S. aureus (MRSA) after 5 minutes of exposure. Data showed no significant effect of KA against MRSA adherent cells. There was strong effect of Formulation O and Comparative Formulations containing 150 ppm AFC on adherent cells after 5 minutes of exposure (reduction >2 log₁₀). Regrowth was observed six hours after the end of treatment with Formulation O and Comparative Formulation. After 5 minutes of exposure to Formulation O and Comparative Formulations, the concentration of adherent bacterial cells (sessile cells) measured 24 hours later were lower than those obtained after the treatment with saline solution.

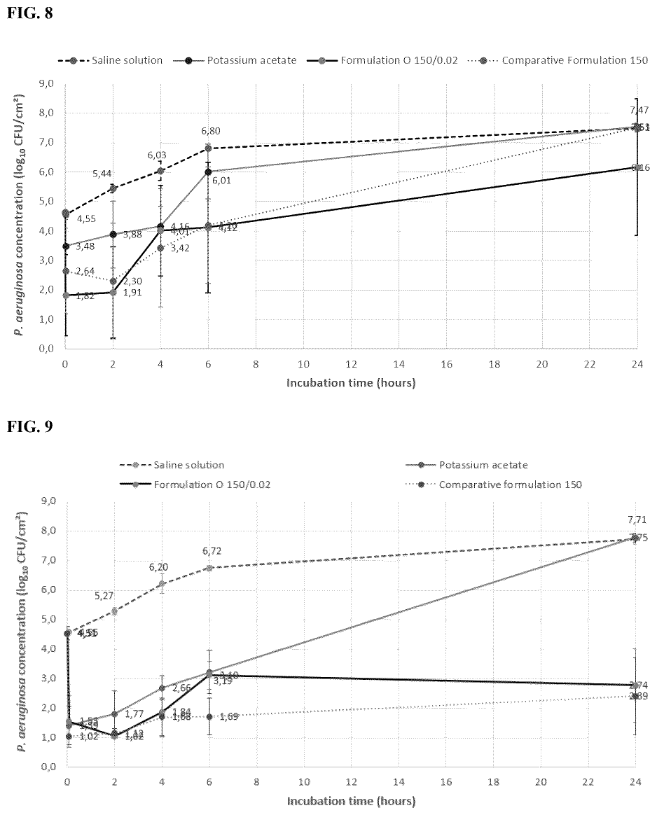

FIG. 8 shows the activity against P. aeruginosa after 1 minute of exposure (3 independent assays). Data showed strong effect of KA against P. aeruginosa adherent cells (decrease). Regrowth was observed 4 hours after the end of treatment for 1 minute with KA. There was bactericidal effect of Formulation O and Comparative Formulations containing 150 ppm AFC on adherent cells after 1 minute of exposure. Regrowth was observed after 2 hours of incubation after treatment with Formulation O and Comparative Formulations containing 150 ppm AFC.

FIG. 9 shows the activity against P. aeruginosa after 5 minutes of exposure (3 independent assays). Data showed strong effect of KA against P. aeruginosa adherent cells (strong decrease of adherent cells). Regrowth was observed after KA treatment between 4 and 24 hours of incubation. There was strong bactericidal effect of Formulation O and Comparative Formulations containing 150 ppm AFC on adherent cells after 5 minutes of exposure (reduction >3 log₁₀). No significant difference was observed between Formulation O and Comparative Formulations. Low regrowth was observed after 24 hours of incubation after treatment with Formulation O and Comparative Formulations containing 150 ppm AFC.

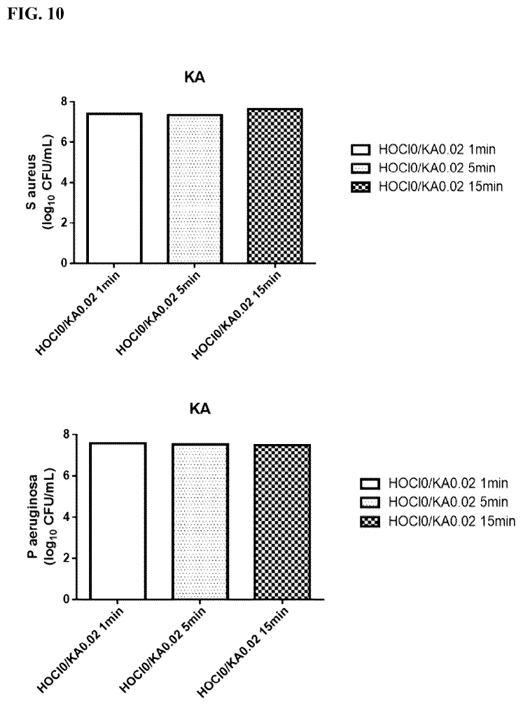

The tests conducted on planktonic cells in the presence of interference substances have demonstrated that Formulation O containing 150 ppm of AFC and 0.02 M of KA was unexpectedly more effective (more pronounced and rapid effect) against P. aeruginosa and S. aureus (MRSA) than the Comparative Formulation containing 150 ppm AFC. No significant effect of KA on the lag-growth phase of planktonic cells after 1, 5, and 15 minutes of exposure was observed (FIG. 10). No significant effect of KA on adherent cells of S. aureus (MRSA) was observed. Strong effects of KA were observed on adherent cells of P. aeruginosa. The concentration of adherent cells was significantly reduced after 1 and 5 minutes of exposure. The Formulation O and Comparative Formulation containing 150 ppm of AFC reduced the concentration of adherent cells after 1 and 5 minutes of exposure and prevented the formation of mature adherent cells.

### Example 3: Evaluation of the Effect of Repeated Applications of Solutions on Mature Biofilms

### Biofilm Model Protocol

Bacterial suspension was prepared in TSB (10⁷ CFU/mL) and 0.5 mL of the prepared bacterial suspension was inoculated on Collagen I-coated microplates and incubated at 37° C. for 24 hours. The medium was removed from the microplate and biofilm was washed twice with saline solution to remove non-adhered bacterial cells. 0.5 mL of tested solutions or saline solution (control) were added. The microplate was incubated at room temperature for 5 minutes. The enumeration of sessile cells was performed before and after exposure time.

After the first treatment, the solutions were removed, and the biofilm was washed twice with saline solution. 0.5 mL of fresh TSB medium was added, and the microplate was incubated at 37° C. for 24 hours. Enumeration of sessile cells after 24 h of regrowth. This process is repeated once to obtain two treatments and follow the kinetics during 48 h.

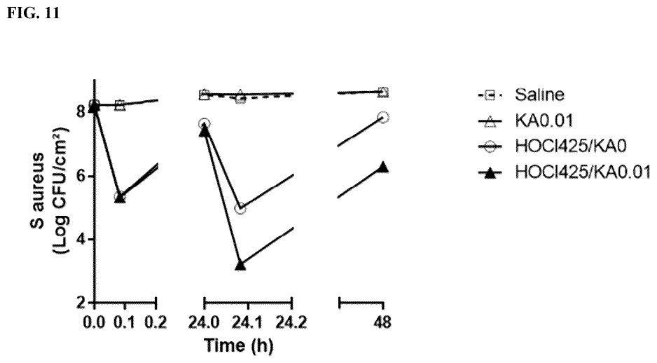

FIG. 11 shows the activity against S. aureus (MRSA) biofilm after 5 minutes of single and repeated exposure. Data showed no significant effect of KA alone against S. aureus (MRSA) biofilm after one or two treatments, similarly to saline solution. There was strong and similar effect of Formulation O and Comparative Formulations containing 425 ppm AFC on S. aureus (MRSA) biofilm after the first application of 5 minutes of exposure with approximately a 3 log₁₀ CFU/cm² reduction; an increase of nearly 2 log 10 CFU/cm² was measured 24 hours later in both groups, but still 1 log₁₀ CFU/cm² lower than in the saline treated group.

After application of the second treatment, a similar effect on the MRSA biofilm to the one observed after the first application was obtained after 5 minutes of contact and 24 hours later.

After application of the second treatment, a more pronounced antimicrobial effect was observed with formulation O with a 5 log₁₀ CFU/cm² reduction compared to the 3 log₁₀ CFU/cm² reduction observed after the first treatment, demonstrating a synergistic effect of HOCl and KA. Moreover, this improved effect was still observed 24 hours later where the log₁₀ CFU/cm² count was 6.3 in the formulation O group, 7.85 in the Comparative Formulation containing 425 ppm AFC, and approximately 8.6 in the saline and KA treated groups.

### Example 4: Evaluation of the Antibiofilm Activity of Solutions Against Mature Bacterial Biofilms

### In Vitro Biofilm Model (Static)

Bacterial suspension was prepared in TSB (10⁷ CFU/mL) and 0.5 mL of the preparation was inoculated on Collagen I-coated microplates and incubated at 37° C. for 24 hours. The medium was removed from the microplate, and the biofilm was washed twice with saline solution to remove non-adhered bacterial cells. 0.5 mL of tested solutions at 4.5 pH or saline solution (control) were added. The microplate was incubated at room temperature for 5 or 15 minutes.

The solutions were removed, and the biofilm cells were washed twice with saline solution. 0.5 mL of sterile TSB was added, and the microplate was incubated at 37° C. Enumeration of sessile cells was performed before exposure (initial biofilm) and immediately after exposure for 5 min and 15 min. Agar plates were incubated at 37° C. for 24 to 48 hours for the recovery of stressed cells.

| | | AFC concentration | Concentration |
|---|---|---|---|
| | Solutions | (ppm) | buffer (M) |
| Potassium | Formulation 1 | 500 | 0.01 KA |
| acetate buffer | Formulation 2 | 425 | 0.01 KA |
| (KA) | Formulation 3 | 500 | 0.005 KA |
| | Formulation 4 | 450 | 0.005 KA |
| Sodium acetate | Formulation 5 | 500 | 0.01 NaA |
| buffer (NaA) | Formulation 6 | 450 | 0.01 NaA |

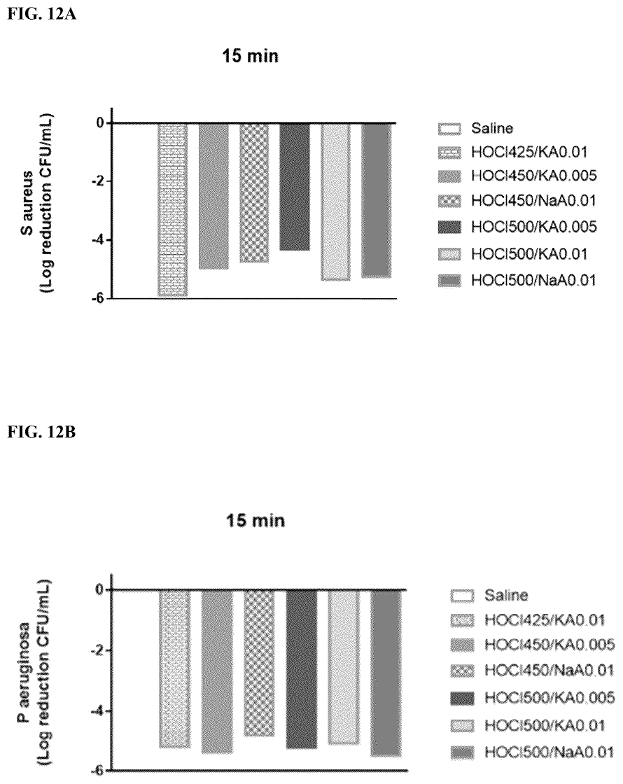

FIG. 12A-B show the activity against S.aureus (MRSA) and P. aeruginosa after 15 minutes of exposure. There was strong antibiofilm effect of all Formulations (reduction >5 log₁₀). No significant difference was observed between Formulation with KA or NaA. The combination of 425 to 500 ppm AFC with KA or NaA were shown to be similarly effective in the eradication of a biofilm.

### Example 5: Evaluation of Cytotoxicity Potential

Mammalian L-929 mouse fibroblast cells were propagated and maintained in vented flasks with EMEM10 at 37° C. (humidified) with 5% carbon dioxide (CO₂). Cells were harvested with trypsin-EDTA solution and counted, then 6 wells plates were seeded with 2.2 ×10⁵ cells, and incubated at 37±1° C. in 5±1% CO₂ to obtain sub-confluent monolayers of cells prior to use. The wells dishes were emptied and the culture medium was replaced by a culture medium-agarose mixture. The agarose mixture was allowed under laminar flow hood to solidify over the cells to form the agarose overlay.

A filter of 1 cm² was saturated with (1) Negative control solution (saline solution), (2) Positive control solution, (3) HOCl composition was applied on agarose surface and incubated at 37±1° C. in 5±1% CO₂ for 24-26 hours. Then the filters were removed of the agarose surface and the cell cultures were stained by a neutral red solution and incubated for 1 hour to 1 hour 30 at 37±1° C. in 5±1% CO₂. The cell cultures were macroscopically examined for cell decolorization around the filters to determine the zone of the cell lysis, if any. After macroscopic examination, the cell monolayer was microscopically examined to verify any decolorized zones and to determine cell morphology in proximity and beneath the filters. A cytotoxic grade based on the severity of the response (Grades 0 for no reactivity to 4 for high reactivity) was assigned according to ISO 10993-5 standard.

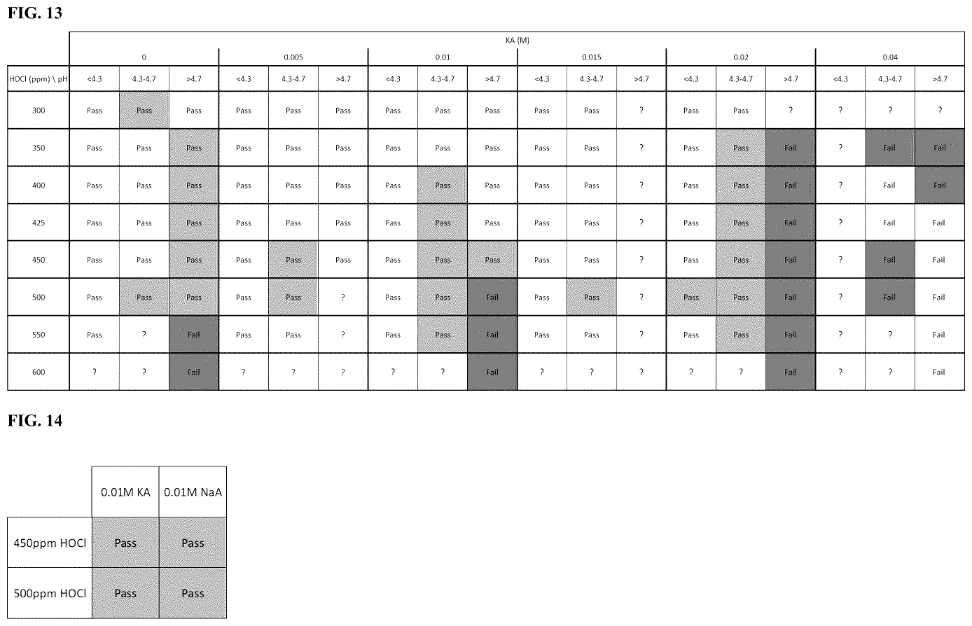

FIG. 13 shows cytotoxicity of HOCl compositions comprising KA on L-929 mouse fibroblasts. Light shaded squares as well as squares labeled “pass” indicate that the formulation is non-cytotoxic; conversely, darker shaded squares and squares labeled “fail” indicate that the formulation exhibited cytotoxicity. The symbol “?” means that we have not tested these formulations.

FIG. 14 show cytotoxicity of HOCl compositions comprising KA or NaA on mouse fibroblasts. Light shaded squares as well as squares labeled “pass” indicate that the formulation is non-cytotoxic.

All references cited herein are incorporated by reference in their entireties.

What is claimed is:

1. An antimicrobial composition comprising hypohalous acid and a salt of an organic acid, wherein the pH of the composition is from about 3.5 to about 6.8, and wherein the salt optionally comprises an acetate salt, and where the acetate salt is optionally potassium acetate and/or sodium acetate.

2. The composition of claim 1, wherein the salt of an organic acid comprises one or more salts of active metals.

3. The composition of claim 1, wherein the salt of an organic acid comprises one or more salts of alkaline, alkaline-earth, and aluminum metals, or is an ammonium salt.

4. The composition of claim 1, wherein the salt is an acetate salt comprising one or more of potassium acetate, sodium acetate, lithium acetate, magnesium acetate, calcium acetate, aluminum acetate, and ammonium acetate.

5.-6. (canceled)

7. The composition of claim 1, wherein the hypohalous acid is selected from hypochlorous acid and hypobromous acid.

8. (canceled)

9. The composition of claim 1, wherein the composition comprises an available free chlorine content (AFC) from about 50 to about 5,000 ppm.

10.-13. (canceled)

14. The composition of claim 1, wherein the salt of an organic acid has a concentration from about 0.001 M to about 1.000 M.

15.-17. (canceled)

18. The composition of claim 1, comprising from about 0.005 M to about 0.02 M potassium acetate and/or sodium acetate, and from about 100 to about 500 ppm hypochlorous acid and optionally from about 400 ppm to about 500 ppm HOCl.

19. The composition of claim 1, wherein the pH is from about 3.8 to about 6.5.

20.-21. (canceled)

22. The composition of claim 1, further comprising a stabilizing amount of dissolved inorganic carbon (DIC).

23. The composition of claim 22, wherein the DIC is incorporated in the form of a bicarbonate or carbonate of an alkali or alkaline earth metal.

24. The composition of claim 23, further comprising a stabilizing amount of sodium bicarbonate.

25. The composition of claim 1, wherein the composition is formulated as a solution, gel, cream, or foam.

26. (canceled)

27. A method for preparing the composition of claim 1, comprising:
incorporating the salt of an organic acid into a hypohalous acid composition, wherein the salt is added to an electrolyte prior to electrolysis or to a hypohalous acid solution after electrolysis.

28. A method for disinfecting or cleansing a mammalian or animal tissue, comprising applying the composition of claim 1 to the mammalian or animal tissue, optionally wherein the tissue is human tissue.

29. The method of claim 28, wherein the mammalian or animal tissue is infected or colonized by bacterial or fungal pathogens, and optionally exhibits biofilm.

30.-33. (canceled)

34. The method of claim 28, wherein the composition is applied topically to the tissue, or to the environment via fogging or misting.

35. A method for disinfecting or cleansing a hard surface, comprising applying the composition of claim 1 to the hard surface.

36. (canceled)

37. A method for preserving or decontaminating food products, comprising applying the composition of claim 1 to a food product or plant product.

38. A method for controlling microbial contamination of water, comprising applying the composition claim 1 to water in need of treatment or control for microbial contamination.
